(19) United States
(12) Patent Application Publication
(10) Pub. No.: US 2013/0263865 A1
(43) Pub. Date: Oct. 10, 2013

(54) SLEEP APNEA PREVENTION ASSEMBLY

(71) Applicant: Julian Omidi, (US)

(72) Inventor: Kaveh Khast, Beverly Hills, CA (US)

(73) Assignee: Julian Omidi, Beverty Hills, CA (US)

(21) Appl. No.: 13/857,937

(22) Filed: Apr. 5, 2013

Related U.S. Application Data
(60) Provisional application No. 61/686,413, filed on Apr. 5, 2012.
(60) Provisional application No. 61/718,662, filed on Oct. 25, 2012.
(60) Provisional application No. 61/773,369, filed on Mar. 6, 2013.

(51) Int. Cl. A61F 5/56

(52) U.S. Cl. CPC A61F 5/566
USPC 128/848

(57) ABSTRACT

A sleep apnea prevention assembly that includes an oral appliance that includes upper and lower members that are hingedly attached to one another and movable between an open position and a closed position. The upper and lower members cooperate to define a central opening when the oral appliance is in the closed position. A flexible lip seal extends between the upper and lower members and the assembly includes a main pipe assembly that comprises a main pipe and a mouth air vent. The main pipe is secured to one of the upper or lower members and extends through the central opening. The mouth air vent has a breathing opening at a proximal end of the main pipe assembly. The main pipe assembly includes an elbow receiver opening at a distal end thereof, and a mouth air path is defined between the breathing opening and the elbow receiver opening.

### CROSS REFERENCE TO RELATED APPLICATIONS

This application claims the benefit of U.S. Provisional Application Nos. 61/686,413, filed Apr. 5, 2012, 61/718,662, filed Oct. 25, 2012, and 61/773,369, filed Mar. 6, 2013, which are each incorporated herein by reference in their entireties.

### FIELD OF THE INVENTION

The present invention relates to a device for the prevention of sleep apnea.

### BACKGROUND OF THE INVENTION

Sleep apnea is a common problem for many people. Devices have been devised for helping prevent sleep apnea. For example, see U.S. Patent Pub. No. 2008/0173313 and U.S. Pat. Nos. 5,117,816 and 5,941,247, the entireties of which are incorporated herein by this reference.

### SUMMARY OF THE PREFERRED EMBODIMENTS

In accordance with a first aspect of the present invention there is provided an oral appliance that includes an upper member that includes an upper teeth receiving channel, and a lower member that includes a lower teeth receiving channel. One of the upper member and the lower member includes right and left hinge members extending rearwardly therefrom. The right and left hinge members each include at least first and second openings defined therein. The other of the upper member and lower member includes right and left posts extending therefrom. The right post is received in the first opening in the right hinge member and the left post is received in the first opening in the left hinge member, thereby securing the upper member to the lower member. The positioning of the upper member with respect to the lower member can be adjusted by removing the right and left posts from the first openings and inserting the right and left posts into the second openings. In a preferred embodiment, the upper member includes an upper base and an upper overmold that are separable from one another and the lower member includes lower base and a lower overmold that are separable from one another, and the right and left hinge members extend rearwardly from one of the upper base or the lower base. Preferably, the upper overmold and the lower overmold are comprised of a material that has a lower softening point than the material of which the upper base, the lower base and the right and left hinge members are comprised. In a preferred embodiment, the upper overmold includes an upper protrusion extending downwardly therefrom that is received in an upper trough defined in the upper base. The upper protrusion comprises a lip and a channel and the upper trough comprises a complementary lip and channel. The lip of the upper protrusion is received in the channel of the upper trough and the lip of the upper trough is received in the channel of the upper protrusion. Preferably, the lower overmold includes a lower protrusion extending upwardly therefrom that is received in a lower trough defined in the lower base. The lower protrusion comprises a lip and a channel and the lower trough comprises a complementary lip and channel. The lip of the lower protrusion is received in the channel of the lower trough and the lip of the lower trough is received in the channel of the lower protrusion.

In an embodiment, the right and left hinge members each include at least first, second, third and fourth openings defined therein and the other of the upper member and lower member includes first and second right posts and first and second left posts extending therefrom. The first and second right posts are received in two of the openings in the right hinge member and the first and second left posts are received in two of the openings in the left hinge member, thereby securing the upper member to the lower member. The positioning of the upper member with respect to the lower member can be adjusted by removing the first and second right posts and first and second left posts from the respective openings and inserting the first and second right posts and first and second left posts into different openings. In a preferred embodiment, the first and second right posts and the first and second left posts each include a flange extending outwardly therefrom. Preferably, the right and left hinge members extend rearwardly from the upper base and wherein the first and second right posts and the first and second left posts extend upwardly from the lower base. In a preferred embodiment, the upper member and the lower member each include a recess defined therein that cooperate to define a central opening that is adapted to receive a portion of a CPAP device (e.g., a pipe, as described herein).

In accordance with another aspect of the present invention, there is provided a sleep apnea prevention assembly that includes an oral appliance that includes an upper member that includes an upper teeth receiving channel, and a lower member that includes a lower teeth receiving channel. The upper and lower members are hingedly attached to one another such that the upper and lower members are movable between an open position and a closed position. The upper member and the lower member each include a recess defined therein that cooperate to define a central opening when the oral appliance is in the closed position. The oral appliance also includes a flexible lip seal that extends between the upper member and the lower member, and a main pipe assembly that comprises a main pipe and a mouth air vent. The main pipe is secured to one of the upper or the lower member and extends through the central opening when the oral appliance is in the closed position. The mouth air vent is positioned proximal of the central opening and has a breathing opening at a proximal end of the main pipe assembly. The main pipe assembly includes an elbow receiver opening at a distal end thereof, and a mouth air path is defined between the breathing opening and the elbow receiver opening. In a preferred embodiment, the flexible lip seal is secured to the upper and lower members by an o-ring that is received in a trough that is defined in the upper member, the lower member and the main pipe.

In a preferred embodiment, the sleep apnea prevention assembly further includes a right nose tube assembly having a right nostril opening, and a left nose tube assembly having a left nostril opening. The right and left nose tube assemblies are secured to and in fluid communication with the main pipe assembly. A right nostril air path extends from the right nostril opening through a first aperture in he main pipe and to the elbow receiver opening, and a left nostril air path extends from the left nostril opening through a second aperture in the main pipe and to the elbow receiver opening. Preferably, the main pipe includes right and left extension members that include right and left circular air openings that are defined by a plurality of extensions that extend between the main pipe and right and left cylindrical receiver members. The right nose tube assembly includes a right lower nose tube that is received on the right extension member and a right upper nose tube in which the right nostril opening is defined extending upwardly from the right lower nose tube. The left nose tube assembly includes a left lower nose tube that is received on the left extension member and a left upper nose tube in which the left nostril opening is defined extending upwardly from the left lower nose tube. In this embodiment, the right nostril air path extends from the right nostril opening, through the right upper nose tube, through the right lower nose tube, through the right circular air opening, through the right aperture and to the elbow receiver opening, and the left nostril air path extends from the left nostril opening, through the left upper nose tube, through the left lower nose tube, through the left circular air opening, through the left aperture and to the elbow receiver opening.

In a preferred embodiment, the right nose tube assembly includes a right valve that adjusts the flow air between the right nostril opening and the right aperture, and the left nose tube assembly includes a left valve that adjusts the flow air between the left nostril opening and the left aperture. Preferably, the right valve includes aright plug that moves axially within the right circular Shaped air opening, and the left valve comprises a left plug that moves axially within the left circular shaped air opening. In a preferred embodiment, the right and left plugs move axially via right and left threaded fasteners, respectively.

In a preferred embodiment, the sleep apnea prevention assembly further includes an elbow assembly adapted to be connected to a CPAP machine. The elbow assembly is secured to the distal end of the main pipe assembly.

The present invention is a device for helping prevent occlusion of a person's airway during sleep. It can be used by people suffering from sleep apnea, snoring issues or both. Generally, the device includes an oral appliance that is inserted into the user's mouth and a connection assembly that is used in conjunction with a continuous positive airway pressure (“CPAP”) machine to provide positive pressure to the back of the oral cavity. The oral appliance can be adjustable (as shown in FIGS. 1-9) or not adjustable (as shown in FIGS. 10-16). In a preferred embodiment, the oral appliance is fitted by a “boil and bite” method, which makes the device easier to be retained within the oral cavity. In preferred embodiment, the oral appliance includes spring action at the hinge area that helps to keep the device in place. In a preferred embodiment, each of the upper and lower members is a two piece plastic device with a base and an overmold. Preferably, the overmold has a lower softening point than the base such that it is readily moldable at a relatively low temperature and the base has a higher softening point so that it retains its spring characteristics when the appliance is placed in hot water. The upper and lower bases also help prevent the upper and lower overmolds from sticking together when placed in hot water and fitted to a user.

It will be understood that the device is intended to be used while the user sleeps and can also be used to help prevent teeth grinding.

The invention, together with additional features and advantages thereof, may be best understood by reference to the following description.

### BRIEF DESCRIPTION OF THE DRAWINGS

FIG. 1 is a perspective view of a sleep apnea prevention device in accordance with a preferred embodiment of the present invention;

FIG. 2 is another perspective view of the sleep apnea prevention device of FIG. 1;

FIG. 3 is a side elevational view of the sleep apnea prevention device of FIG. 1;

FIG. 4 is an exploded side elevational view of the sleep apnea prevention device of FIG. 1;

FIG. 5 is an exploded perspective view of the sleep apnea prevention device of FIG. 1;

FIG. 6 is a front elevational view of the sleep apnea prevention device of FIG. 1;

FIG. 7 is a cross-sectional side elevational view of the sleep apnea prevention device of FIG. 1 showing the complementary lips and channels;

FIG. 8 is a cross-sectional side elevational view of the sleep apnea prevention device of FIG. 1 showing the relationship of the posts and openings;

FIG. 9 is a cross-sectional side elevational view of the sleep apnea prevention device of FIG. 1 showing the relationship of the posts and openings, but with the lower member positioned rearwardly from the position shown in FIG. 8;

FIG. 10 is a perspective view of a sleep apnea prevention assembly in accordance with a preferred embodiment of the present invention;

FIG. 11 is a perspective view of the sleep apnea prevention assembly of FIG. 10 with the nose tube assemblies, oral appliance, elbow assembly and main pipe exploded from one another;

FIG. 12 is an exploded perspective view of the sleep apnea prevention assembly of FIG. 10;

FIG. 13 is a cross-sectional view of the sleep apnea prevention assembly of FIG. 10;

FIG. 14 is a cross-sectional view of the sleep apnea prevention assembly of FIG. 10;

FIG. 15 is a cross-sectional view of the sleep apnea prevention assembly of 10;

FIG. 16 is a perspective view of the sleep apnea prevention assembly of FIG. 10 including a lip seal and with the chin strap rings and elbow omitted;

FIG. 17 is a perspective view of the main pipe, the diffuser therein and with the nose tube assemblies omitted;

FIG. 18 is a perspective view of the nose tube assembly valve in a fully extended position;

FIG. 19 is a perspective view of the nose tube assembly valve in a fully retracted position;

FIG. 20 is a cross-sectional view of a portion of the sleep apnea prevention assembly of FIG. 10 including the lip seal; and

FIG. 21 is a perspective view of a portion of a sleep apnea prevention assembly of showing the lip seal and 0-ring exploded therefrom in accordance with a preferred embodiment of the present invention.

### DETAILED DESCRIPTION OF THE PREFERRED EMBODIMENTS

The following description and drawings are illustrative and are not to be construed as limiting. Numerous specific details are described to provide a thorough understanding of the disclosure. However, in certain instances, well-known or conventional details are not described in order to avoid obscuring the description. References to one or an other embodiment in the present disclosure can be, but not necessarily are, references to the same embodiment; and, such references mean at least one of the embodiments.

Reference in this specification to “one embodiment” or “an embodiment” means that a particular feature, structure, or characteristic described in connection with the embodiment is included in at least one embodiment of the disclosure. Appearances of the phrase “in one embodiment” in various places in the specification do not necessarily refer to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks: The use of highlighting has no influence on the scope and meaning of a term; the scope and meaning of a term is the same, in the same context, whether or not it is highlighted. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further nit the scope of the disclosure, examples of instruments, apparatus, methods and their related results according to the embodiments of the present disclosure are given below. Note that titles or subtitles may be used examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

It will be appreciated that terms such as “front,” “back,” “top,” “bottom,” “side,” “short,” “long,” “up,” “down,” and “below” used herein are merely for ease of description and refer to the orientation of the components as shown in the figures. It should be understood that any orientation of the components described herein is within the scope of the present invention.

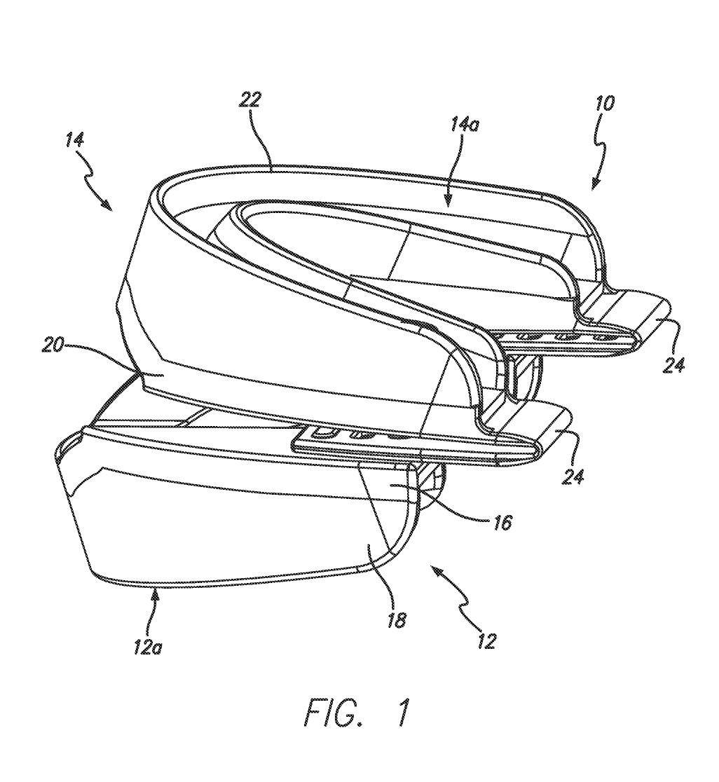

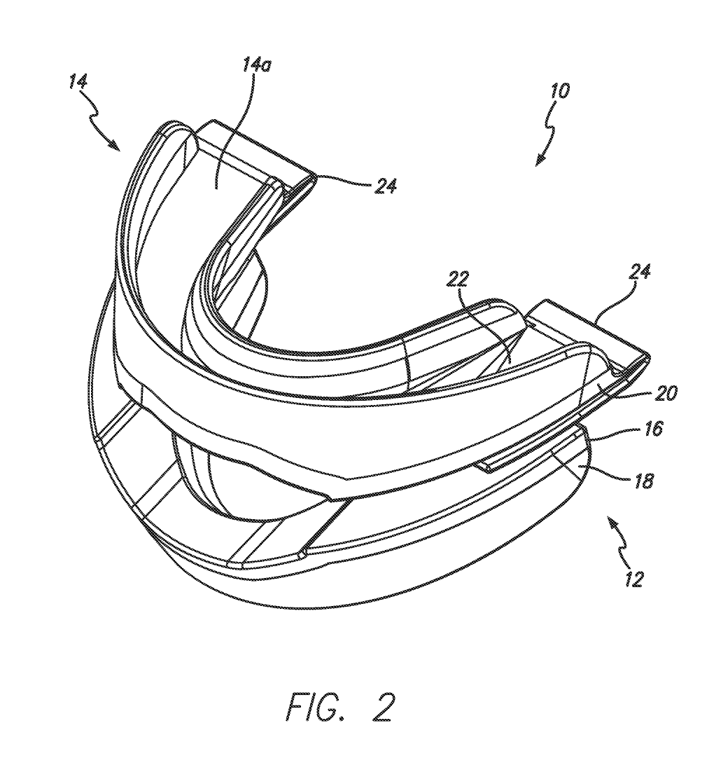

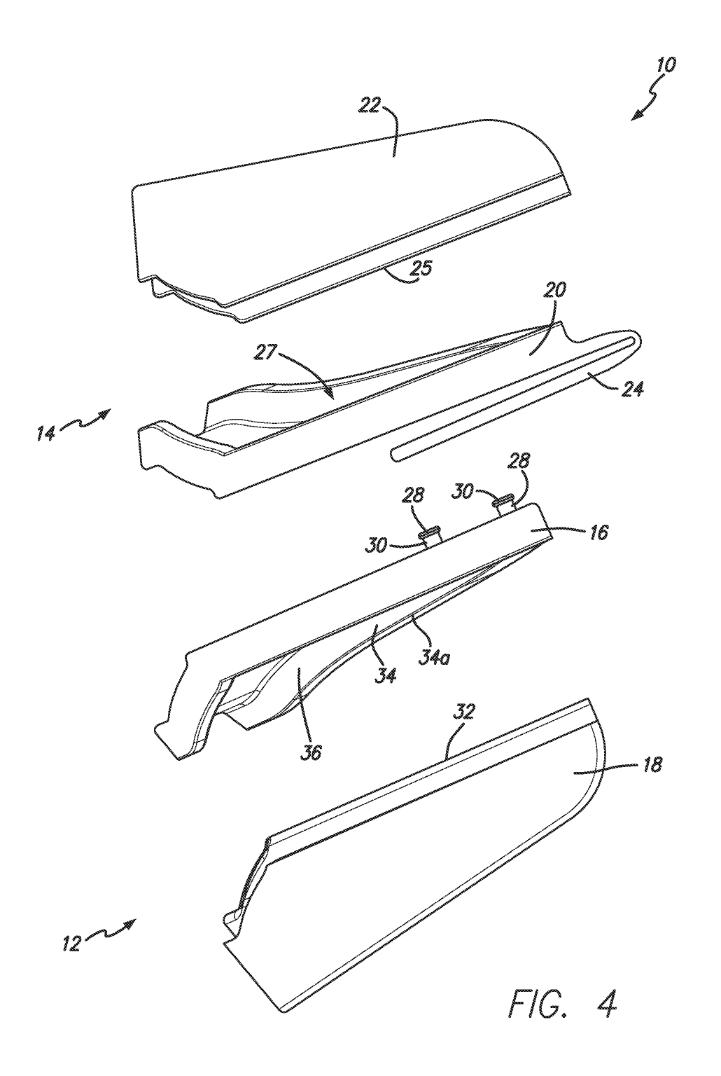

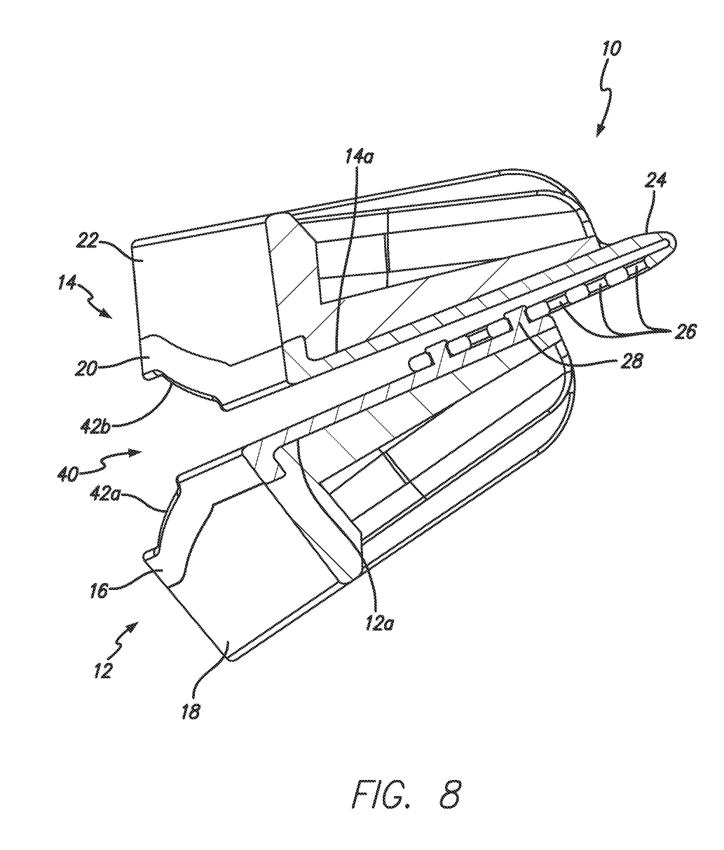

Referring now to the drawings, wherein the showings are for purposes of illustrating the present invention and not for purposes of limiting the same, FIGS. 1-9 show a sleep apnea prevention device, mouthpiece or oral appliance 10. As shown in the figures, oral appliance 10 includes a lower member 12 and an upper member 14. The lower member 12 preferably includes a lower teeth receiving channel 12a and the upper member 14 preferably includes an upper teeth receiving channel 14a. In a preferred embodiment, the lower member 12 includes a lower base 16 and a lower overmold 18. The upper member 14 includes an upper base 20 and an upper overmold 22.

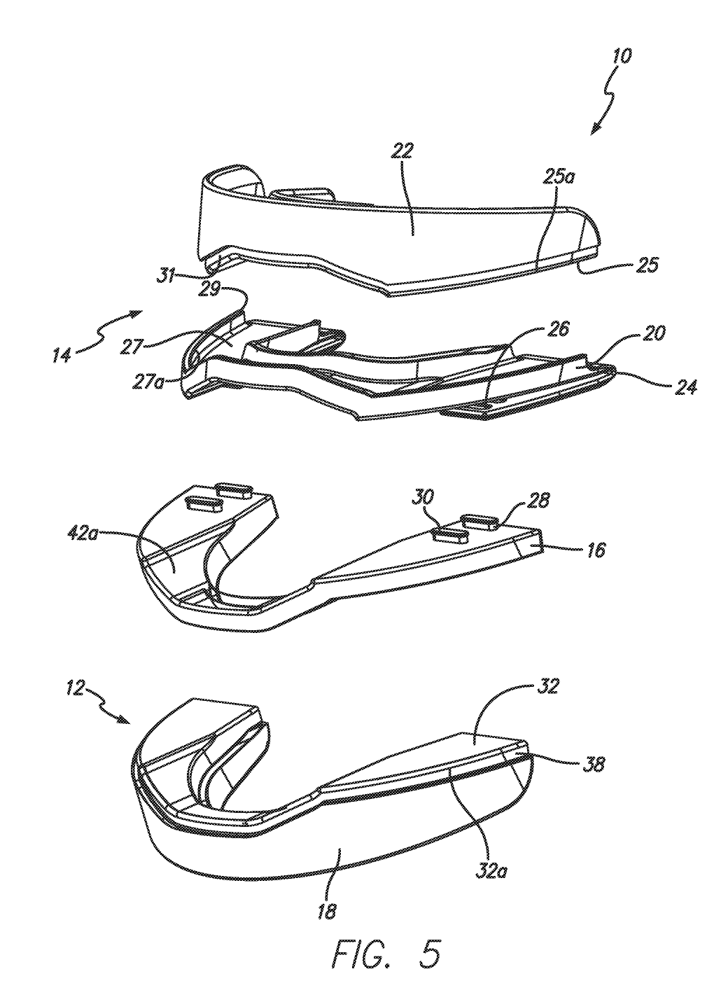

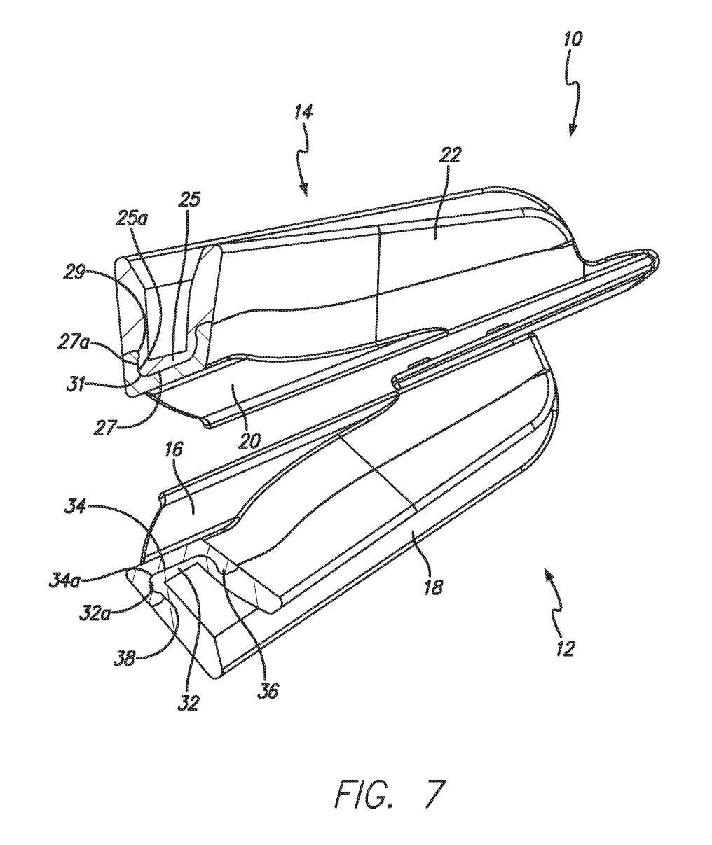

As is best shown in FIG. 5, the upper base 20 includes a pair of hinge members 24 extending rearwardly therefrom. The hinge members 24 each include a plurality of openings 26 therein that are adapted to receive a post 28 and flange 30 that extend upwardly from the lower base 16 (see, e.g., FIGS. 7-9). It will be understood this arrangement can be reversed such that the posts and flanges extend downwardly from the upper base 20 and the hinge members 24 extend rearwardly from the T base 16. When the upper and lower members 14 and 12 are secured together by the hinge members 24, the upper and lower members are movable between a closed position and an open position.

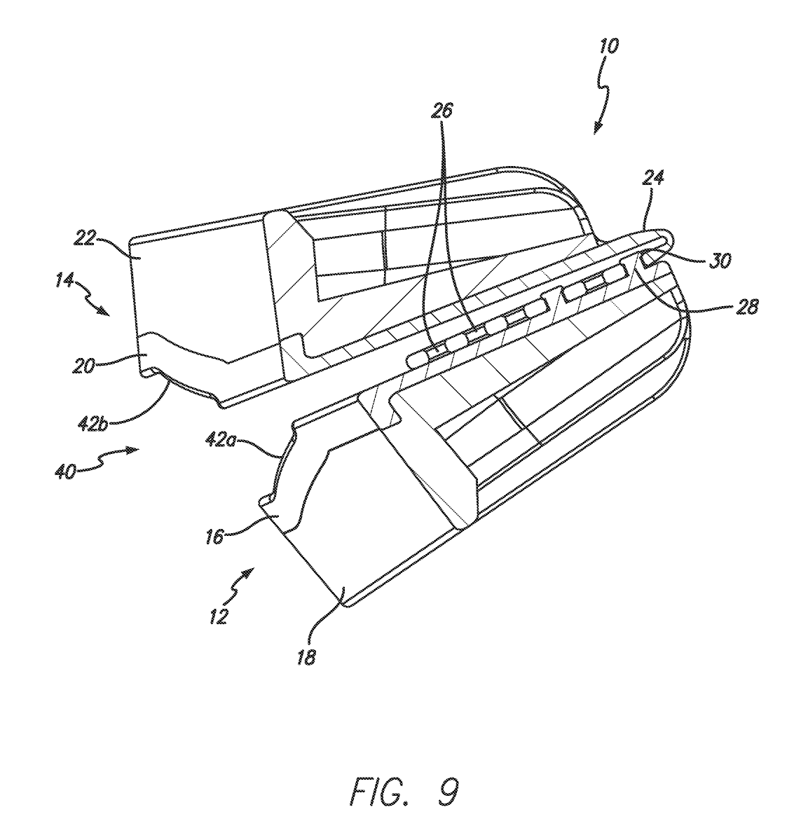

As is shown in FIG. 7, the hinge members 24 include multiple openings 26 therein. This provides for the adjustability of the lower member 12 within the user's mouth. It will be appreciated by those skilled in the art that this arrangement allows the lower member 12 to be moved forwardly rearwardly within the oral cavity to help open the airway as desired. See, e.g., FIG. 9, where the lower member 12 is moved rearwardly when compared to FIG. 7. In a preferred embodiment, the oral appliance 10 provides adjustability between about −4.0 mm and about 5.0 mm within a user's mouth. However, this is not a limitation on the present invention.

In a preferred embodiment, the upper overmold 22 and lower overmold 18 are separable from the upper base 20 and lower base 16, respectively. Preferably, the overmolds 18 and 22, and bases 16 and 20 are in a press fit or snap fit relationship. For example, as shown in FIG. 5, the upper overmold 22 includes an upper protrusion 25 (that comprises a lip 25a and a channel 31) that is received in an upper trough 27 (that comprises a lip 29 and channel 27a) defined in the upper base 20. To secure the upper overmold 22 on the upper base 20, lip 29 is received in channel 31 and lip 25a is received channel 27a. Furthermore, the lower overmold 18 includes a lower protrusion 32 (that comprises a lip 32a and a channel 38) that is received in a lower trough 34 (that comprises a lip 36 and channel 34a) in the lower base 16. To secure the lower overmold 18 on the lower base 16, lip 34 is received in channel 38 and lip 32a is received channel 34a.

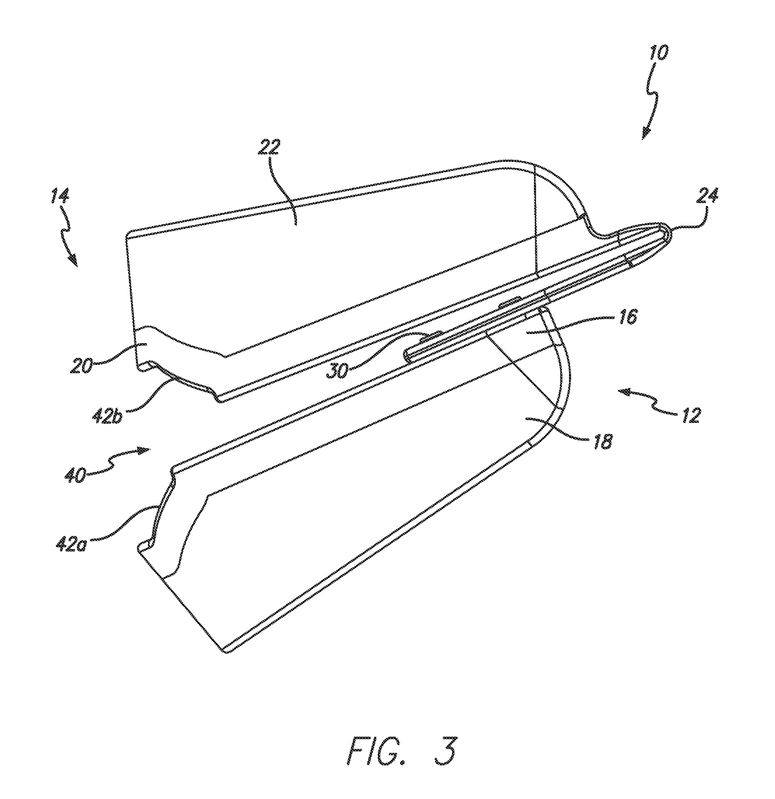

In a preferred embodiment, the hinge members 24 comprise a material that is resilient enough to provide spring action such that the hinge members 24 are normally positioned as shown in FIG. 3 (referred to herein as the “normal position”). In a preferred embodiment, the hinge members 24 provide sufficient spring to hold the oral appliance 10 in the normal position while a user is sleeping. This helps keep the oral appliance 10 in place. However, this is not a limitation on the present invention and the hinge members 24 can be designed so that they do not include any spring action.

In a preferred embodiment, the oral appliance 10 is fitted by a “boil and bite” method. This method includes placing the oral appliance 10 (o c overmolds 18 and 22 separately) into boiling/hot water until the overmolds 18 and 22 soften. The oral appliance 10 is then placed in the users mouth and the user bites down to form indentations in the overmolds 18 and 22 that correspond to the user's teeth. Preferably, the upper and lower overmolds 22 and 18 have a lower softening point than the upper and lower bases 20 and 16 such that the overmolds are moldable at a relatively low temperature and the base has a softening point (melting point) that is high enough so that it (and more specifically, the hinge members 24) will not soften significantly when placed in boiling/hot water, and so that the hinge members 24 will retain their spring characteristics. It will be appreciated by those skilled in the art that by producing the bases 16 and 20 out of a material with a high softening point, it will prevent the bases 16 and 20, and, therefore, the overmolds 1.8 and 22 from sticking together when placed in hot water and fitted to a user.

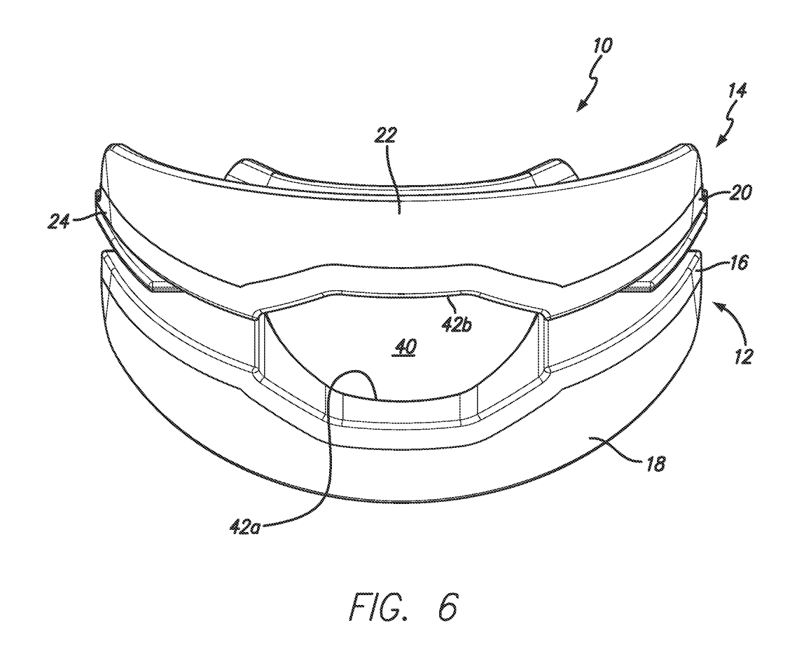

As is best shown in FIG. 6, in preferred embodiment, the oral appliance 10 includes a central opening 40 that is thrilled by recesses 42a and 42b defined in the lower and upper members 12 and 14. The central opening 40 provides for the ability to use the oral appliance 10 in conjunction with a CPAP machine, as described below. However, the central opening 40 can be omitted.

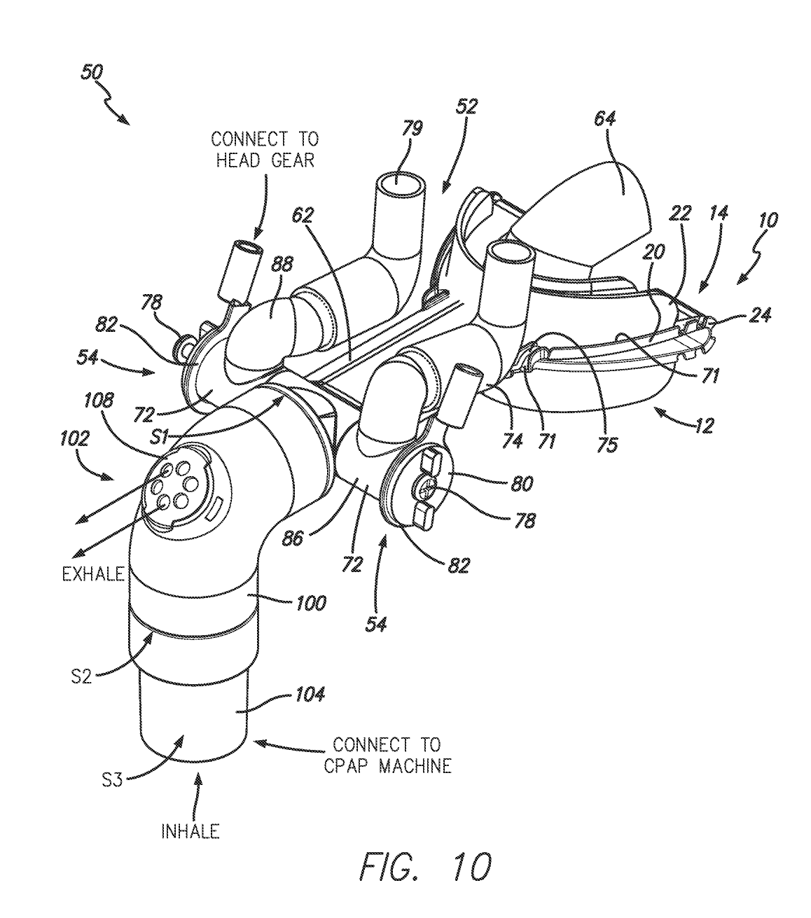

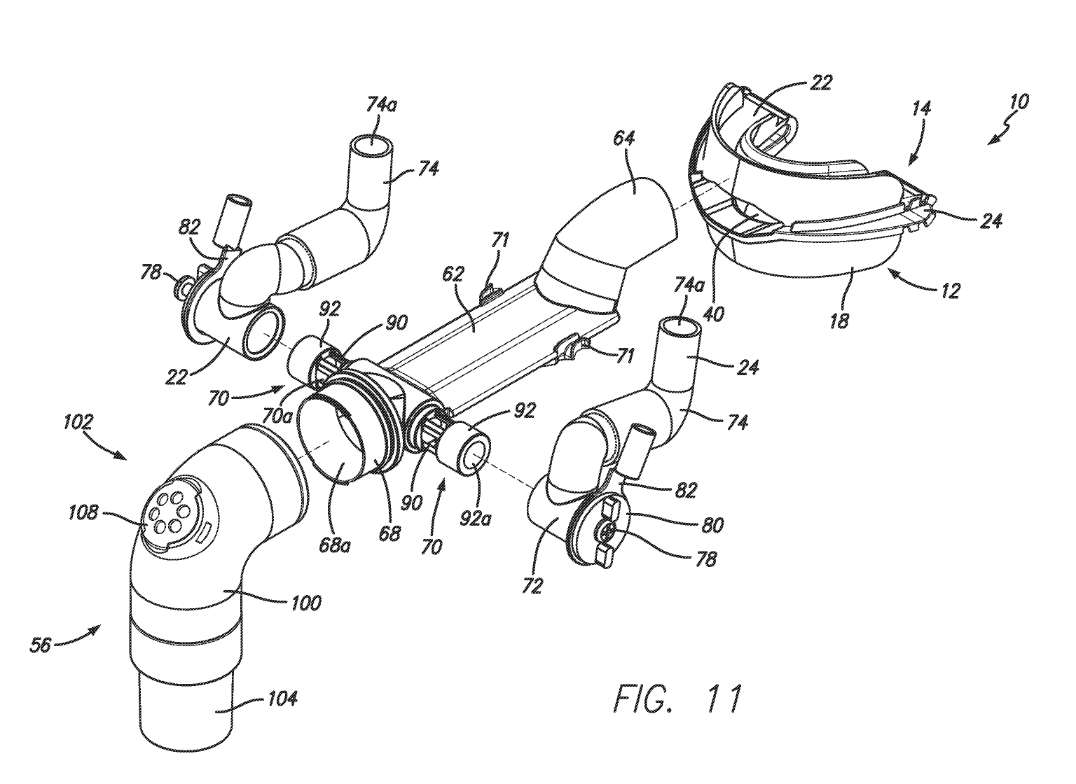

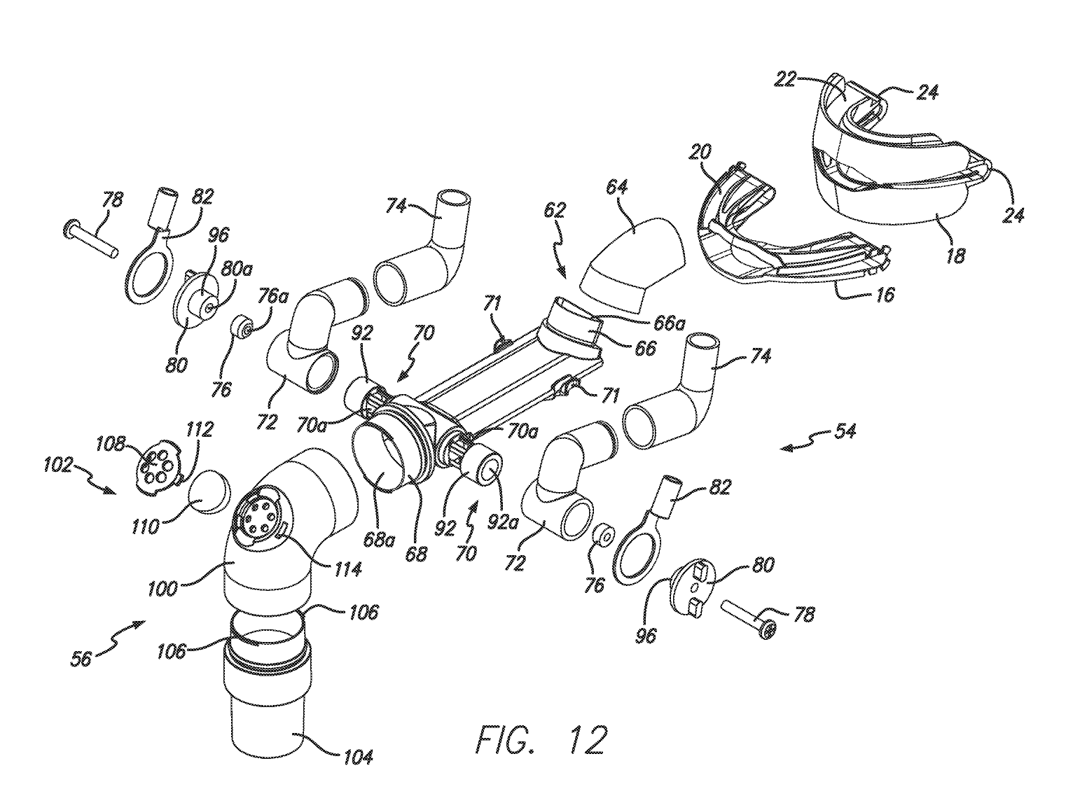

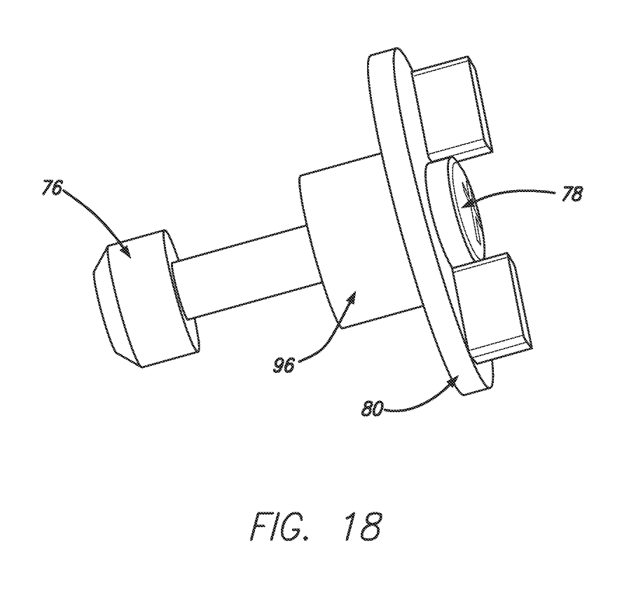

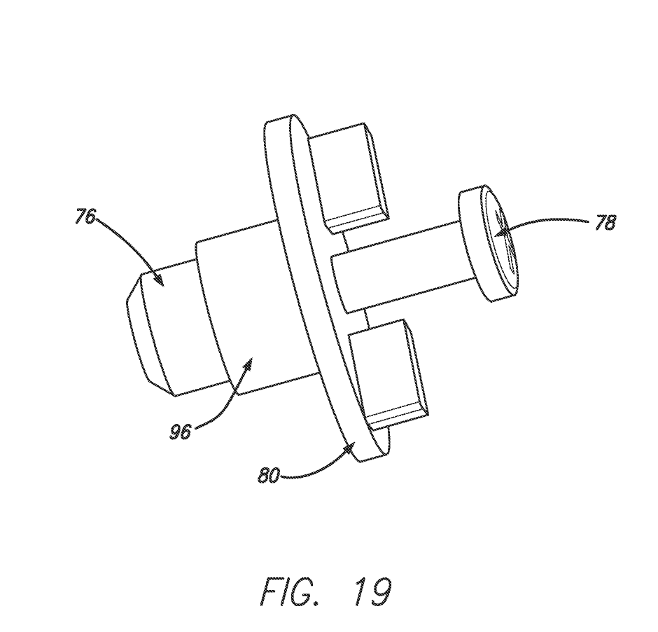

FIGS. 10-21 show a sleep apnea prevention assembly 50, that is used in conjunction with a CPAP machine (not shown). As is best shown in FIGS. 10-11, sleep apnea prevention assembly 50 generally includes oral appliance 10 (the embodiment shown in FIGS. 10-19 does not include the adjustability described above), main pipe assembly 52, left and right nose tube assemblies 54, and elbow assembly 56. As shown in FIG. 12, in an embodiment where the oral appliance 10 is not adjustable, the upper and lower overmolds 18 and 22 and the upper and lower bases 16 and 20 can be unitary pieces.

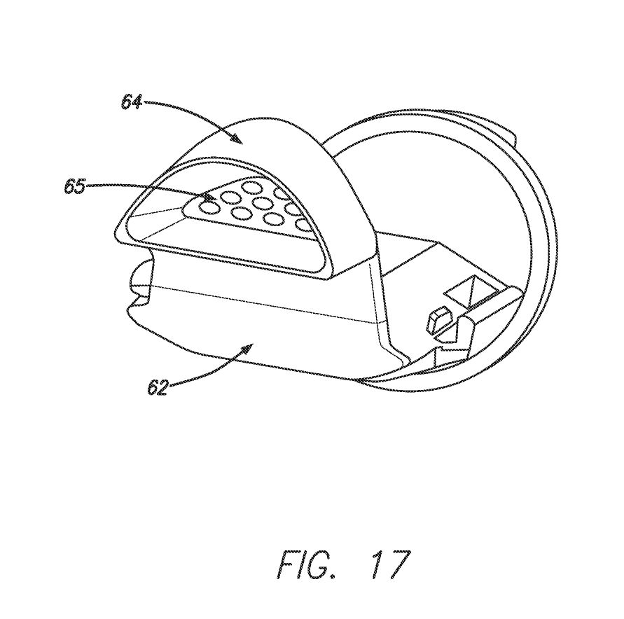

As is shown best in FIG. 12, in a preferred embodiment, the main pipe assembly 52 includes main pipe 62, mouth air vent 64 and, preferably, a diffuser 65 disposed therein (as shown in FIG. 17). In preferred embodiment, main pipe 62 is unitary piece that includes an air vent receiver 66 that includes a vent opening 66a at one end, an elbow receiver 68, having an elbow receiver opening 68a at the opposite end, and left and right extensions members 70, each having a radially extending air opening 70a defined therein, as is shown in FIG. 12. In a preferred embodiment, mouth air vent 64 mates with air vent receiver 66 and elbow assembly 56 mates with elbow receiver 68.

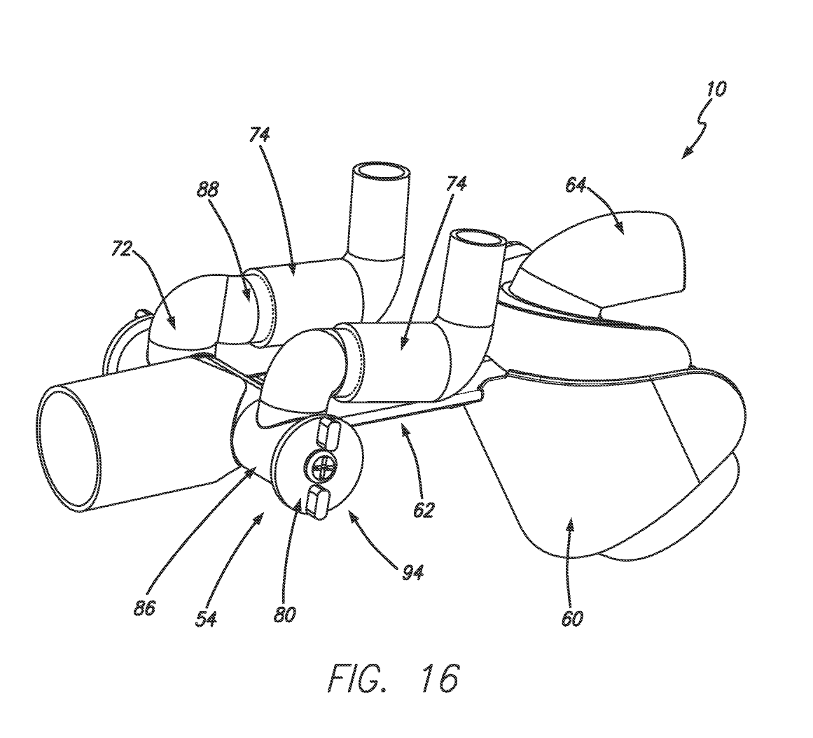

As shown in FIG. 16, in a preferred embodiment, when used with the sleep apnea prevention assembly 50, the oral appliance 10 includes a flexible lip seal 60. Preferably, the flexible lip seal 60 is a thin membrane that is relatively flat when the oral appliance 10 is in the open position (as shown in FIG. 16) and is flexible enough to allow the oral appliance 10 to pivot or hinge to the closed position. The purpose of the flexible lip seal 60 is to prevent air from passing through the opening defined between the upper and lower members 14 and 12, thus forcing air to only pass through the mouth air vent 64. The flexible lip seal 60 can be secured to the lower member 12 and upper member 14 permanently or in a removable fashion and can be secured thereto in any desired way, such as glue, o-rings, snap or press fit arrangements, etc.

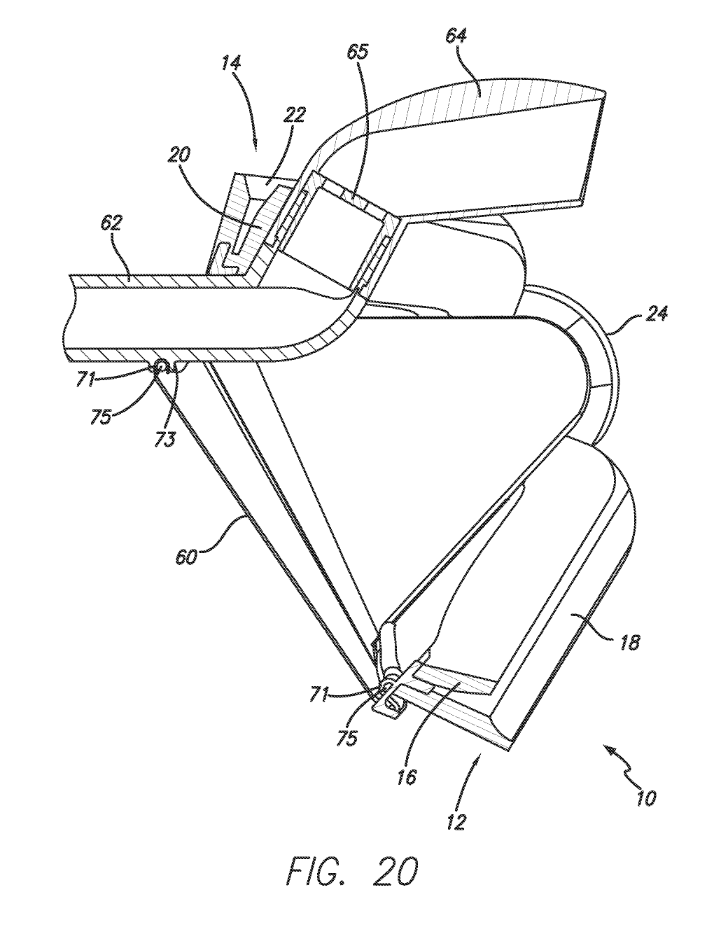

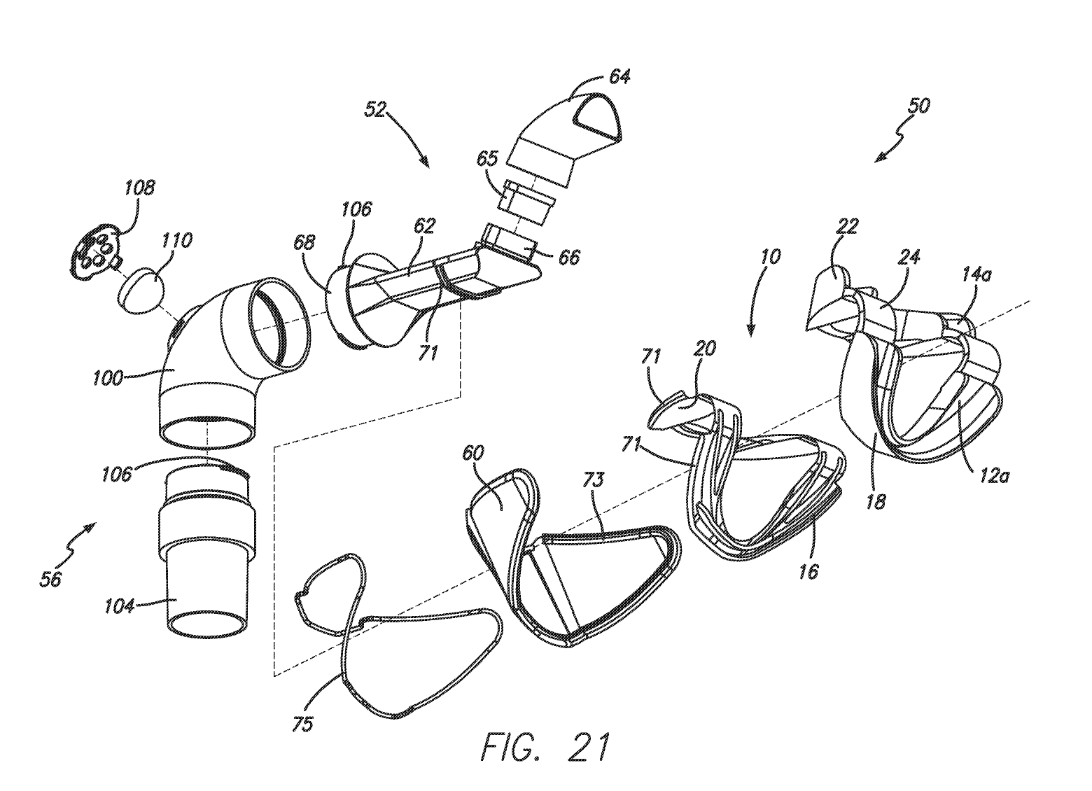

FIGS. 16 and 20-21 show an example of how the flexible lip seal 60 can be secured to the lower and upper members 12 and 14. As shown in FIGS. 20-21, in a preferred embodiment, the lower and upper members 12 and 14 and the main pipe 62 on the underside thereof) include a channel 71. The outer edge of the flexible lip seal 60 (which may comprise a flange 73) is received or seated in the channel 71, and an elastic band or o-ring 75 is pressed into the channel 71 to retain the outer edge of the flexible lip seal 60 therein. In a preferred embodiment, a portion of the channel 71 is formed in the underside and sides (see FIG. 11) of the main pipe 62, a portion is formed in the upper base 20 and a portion is formed in the lower base 16. It will be appreciated that trough 71 can be formed as a depression in the upper base 20, lower base 16 or main pipe 62 or can be defined by ribs that protrude from the upper base 20, lower base 16 or main pipe 62 or a combination of both. FIG. 21 shows an embodiment of the sleep apnea prevention assembly 50 with the left and right nose assemblies omitted.

The left and right nose tube assemblies 54 are virtually identical, but are mirror images of one another. It will be appreciated that any description of one of the components of the left and right nose tube assemblies applies equally to the other of the left and right nose tube assemblies. Therefore, the terms “right” and “left” are omitted in some portions of the description. In a preferred embodiment, the left and right nose tube assemblies 54 each include a lower nose tube 72, upper nose tube 74, plug 76, threaded fastener 78, cap 80 and chin strap ring 82. The lower nose tube 72 includes a cylindrical portion 86 and an angled portion 88. As s own in the figures, the cylindrical portion 86 is received on the extension member 70.

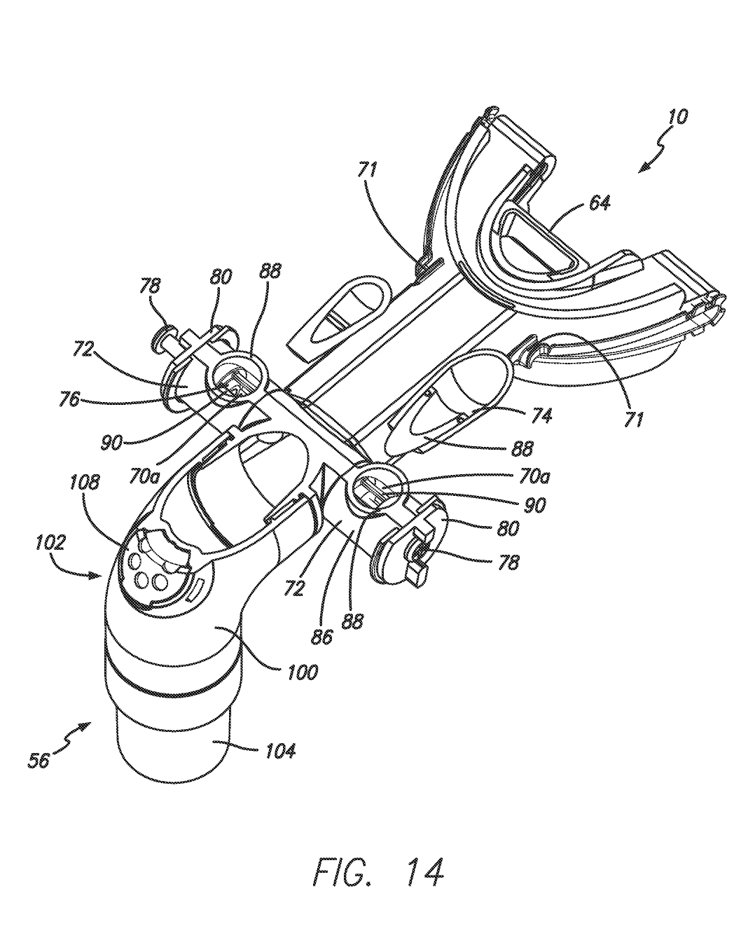

As is best shown in FIGS. 12 and 14-15, right and left air openings 70a are defined by a plurality of extensions 90 that extend between the main pipe 62 and a receiver member 92. The extensions 90 allow the main pipe 62 and the right and left extension members 70 to be in fluid communication with the right and left nose tube assemblies 54 as a result of the cylindrical shape of the cylindrical portion 86 and the circular shaped air opening 70a, as is shown in FIG. 14.

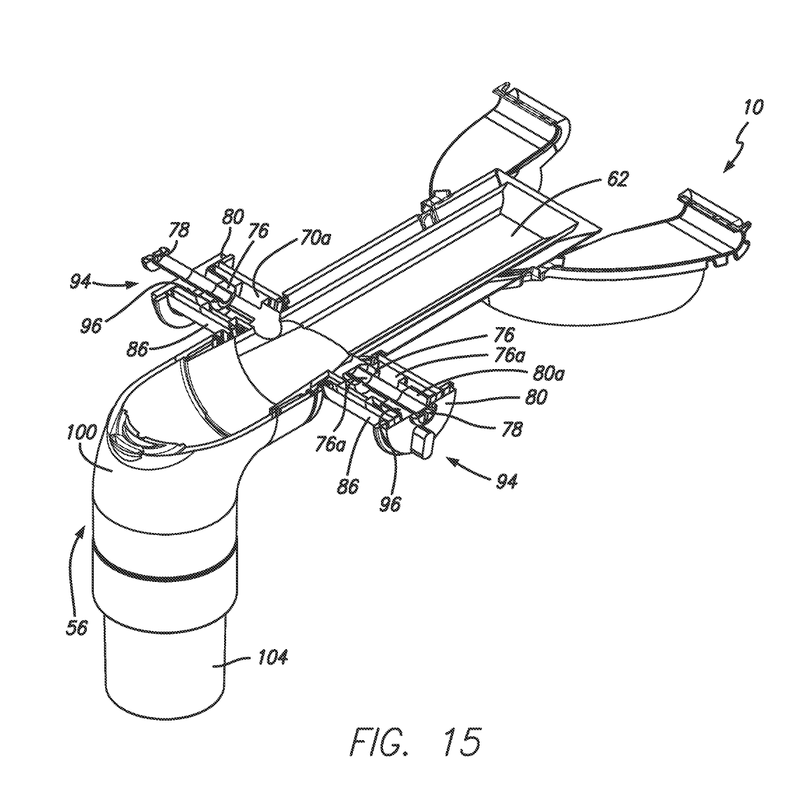

In a preferred embodiment, the sleep apnea prevention assembly 50 includes right and left valves 94 that allow adjustability of the amount of air that flows into and/or out of the right and left nose tube assemblies 54. Any type of valve is within the scope of the present invention. An exemplary valve 94 is shown in FIGS. 14-15. Left and right receiver members 92 each include a threaded interior opening 92a that receives an exteriorly threaded member 96 on the cap 80, to secure the cap 80 in place. Threaded fastener 78 extends through an internally threaded aperture 80a in the cap and is received in an opening 76a in the plug 76. As shown in FIG. 15, 18 and 19, when threaded fastener 78 is turned, it moves the plug 76 axially within circular shaped air opening 70a. As a result, the plug 76 blocks more or less of the air opening 70a as desired and regulates the amount of air flowing into and out of the right and left nose tube assemblies 54. FIG. 15 shows the right valve 94 in a fully open position, and the left valve 94 in a fully closed position, such that a left aperture 98 between the main pipe 62 and the extension member 70 is blocked. In another embodiment, the valves can be omitted. In this embodiment, threaded interior openings 92a would be omitted and the lower nose tubes would include a closed end be unitary with the cap).

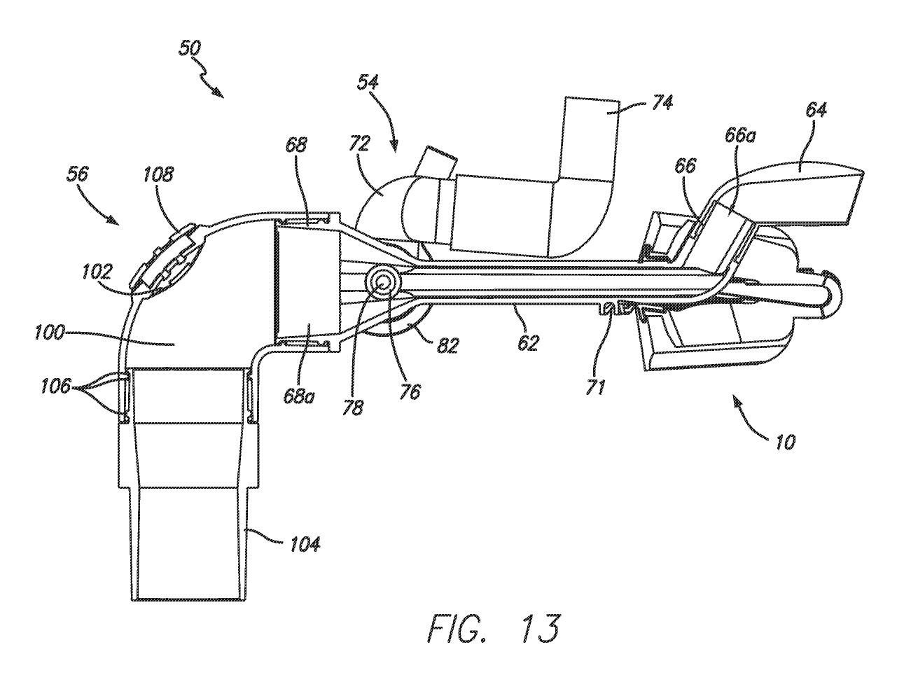

As discussed above, the elbow assembly 56 is used to connect the main pipe 62 to the CPAP machine. In a preferred embodiment, the elbow assembly 56 includes an elbow member 100, vent 102 and lower swivel connector 104. The elbow member 100 and lower swivel connector 104 are hollow and define part of the air path. In another embodiment, the elbow member 100 and lower swivel connector 104 can be a unitary piece. Elbow member 100 is received on and connects with elbow receiver 68. As shown in FIG. 13, in a preferred embodiment, elbow member 100, lower swivel connector 104 and elbow receiver 68 include complimentary circumferentially extending flanges or tabs 106 on interior or exterior surfaces that help keep the components secured to one another, but allow swiveling or rotation with respect to one another, as described below. The tabs 106 can extend circumferentially around the entire component or only partially around, as is shown in FIG. 12. The tabs 106 are made of a malleable material that allows one component to fit over the other in a snap fit arrangement.

In a preferred embodiment 102 is defined in elbow member 100 and includes vent cap 108 and filter 110. In use, as is shown in FIG. 10, user exhaled air exits through vent 102. Any type of vent that allows exhaled air to exit is within the scope of the present invention. Vent cap 108 retains filter 110 in place and includes tabs 112 that fit into complementary openings 114 in the elbow member 100.

In use, the connection between the elbow receiver 68, elbow member 100 and lower swivel connector 104 allows the components to rotate with respect to one another. This is advantageous when a user wears the assembly 50 and moves during the night. The elbow receiver 68 and elbow member 100 can rotate with respect to one another, thereby providing an upper swivel (see arrow S1 in FIG. 10). The elbow member 100 and lower swivel connector 104 can also rotate with respect to one another, thereby providing a lower swivel (see arrow S2 in FIG. 10). The lower swivel connector 104 can also provide for rotation or swiveling with respect to the CPAP machine connected thereto (see arrow S3 in FIG. 10). In another embodiment, the elbow receiver 68, elbow member 100 and lower swivel connector 104 can be permanently connected, unitary or connected to one another such that no rotation is provided. The connection at S1 also allows the elbow assembly 56 to be removable and replaceable with respect to the main pipe assembly 52.

An exemplary use of the present invention will now be described. If a patient has sleep apnea or snoring problems, the patient visits a dentist and is provided with the oral appliance 10, which can be adjusted by the dentist as desired or prescribed. In another embodiment, the patient can purchase the oral appliance 10 at a store. If the oral appliance 10 alone does not solve the problem to the patient or dentist's liking, the sleep apnea prevention assembly 50 can then be provided. The oral appliance 10 previously used can be fitted into the assembly 50 or a new one can be used. As shown in FIG. 10, a portion of the main pipe 62 and the air vent 54 is inserted through central opening 40 in the oral appliance 10 and secured therein. The main pipe 62 can be secured to the upper or lower member 14 or 12 and in any fashion, such as sonic welding, gluing, adhering, etc. Also, the flexible lip seal 60 is secured to the oral appliance 10, so that when the oral appliance 10 is inserted into the patient's oral cavity, air cannot escape through the central opening 60, but only through the breathing opening 64a and mouth air vent 64. To wear the assembly 50, the oral appliance 10 is inserted into the patients oral cavity and the right and left upper nose tubes 74 are inserted into the patient's nostrils. The right and left upper nose tubes 74 each include a nostril opening 74a defined therein. Pillow seals can be used to help maintain a seal between the right and left upper nose tubes 74 and the patient's nostrils and to provide comfort. A chin strap or head strap that extends out of or is otherwise secured to the right and left chin strap rings 82 can be worn by the patient. The chin strap or other head gear can be connected to places other than the chin strap rings 82. The right and left valves 94 can be adjusted (as described above) as desired by the patient and/or the patient's dentist or doctor. As will be appreciated by those skilled in the art, air is inhaled from the connected CPAP machine through the elbow assembly 56, the main pipe 62, the mouth air vent 64 and into the patient's oral cavity. If the right and left valves 94 are open, some air will pass through apertures 98, through extension members 70, through right and left air openings 70a and through right and left lower and upper nose tubes 72 and 74 and into the patient's nostrils. Exhaled air follows the same paths from the patient's oral cavity and nostrils to and through the main pipe 62, but then exits through the vent 102.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to,” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and any particular portions of this application. Where the context permits, words in the above Detailed Description of the Preferred Embodiments using the singular or plural number may also include the plural or singular number respectively. The word “or” in reference to a list of two or more items, covers all of the following interpretations of the word: any of the items in the list, all of the items in the list, and any combination of the items in the list.

The above-detailed description of embodiments of the disclosure is not intended to be exhaustive or to limit the teachings to the precise form disclosed above. While specific embodiments of and examples for the disclosure are described above for illustrative purposes, various equivalent modifications are possible within the scope of the disclosure, as those skilled in the relevant art will recognize. For example, whole processes or blocks are presented in a given order, alternative embodiments may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, or may be performed, at different times. Further any specific numbers noted herein are only examples: alternative implementations may employ differing values or ranges.

The teachings of the disclosure provided herein can be applied to other systems, not necessarily the system described above. The elements and acts of the various embodiments described above can be combined to provide further embodiments.

Any patents and applications and other references noted above, including any that may be listed in accompanying filing papers, are incorporated herein by reference in their entirety. Aspects of the disclosure can be modified, if necessary, to employ the systems, functions, and concepts of the various references described above to provide yet further embodiments of the disclosure.

These and other changes can be made to the disclosure in light of the above Detailed Description of the Preferred Embodiments. While the above description describes certain embodiments of the disclosure, and describes the best mode contemplated, no matter how detailed the above appears in text, the teachings can be practiced in many ways. Details of the system may vary considerably in its implementation details, while being encompassed by the subject matter disclosed herein. As noted above, particular terminology used when describing certain features or aspects of the disclosure should not be taken to imply that the terminology is being redefined herein to be restricted to any specific characteristics, features or aspects of the disclosure with which that terminology is associated. In general, the terms used in the following claims should not be construed to limit the disclosures to the specific embodiments disclosed in the specification unless the above Detailed Description of the Preferred Embodiments section explicitly defines such terms. Accordingly, the actual scope of the disclosure encompasses not only the disclosed embodiments, but also all equivalent ways of practicing or implementing the disclosure under the claims.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention.

What is claimed is:

1. An oral appliance comprising:
an upper member that includes an upper teeth receiving channel, and
a lower member that includes a lower teeth receiving channel,
wherein one of the upper member and the lower member includes right and left hinge members extending rearwardly therefrom, wherein the right and left hinge members each include at least first and second openings defined therein,
wherein the other of the upper member and lower member includes right and left posts extending therefrom, wherein the right post is received in the first opening in the right hinge member and the left post is received in the first opening in the left hinge member, thereby securing the upper member to the lower member,
wherein the positioning of the upper member with respect to the lower member can be adjusted by removing the right and left posts from the first openings and inserting the right and left posts into the second openings.

2. The oral appliance of claim 1 wherein the upper member includes an upper base and an upper overmold that are separable from one another, wherein the lower member includes a lower base and a lower overmold that are separable from one another, and wherein the right and left hinge members extend rearwardly from one of the upper base or the lower base.

3. The oral appliance of claim 2 wherein the upper overmold and the lower overmold are comprised of a material that has a lower softening point than the material of which the upper base, the lower base and the right and left hinge members are comprised.

4. The oral appliance of claim 3 wherein the upper overmold includes an upper protrusion extending downwardly therefrom that is received in an upper trough defined in the upper base, wherein the upper protrusion comprises a lip and a channel and the upper trough comprises a complementary lip and channel, wherein the lip of the upper protrusion is received in the channel of the upper trough and the lip of the upper trough is received in the channel of the upper protrusion.

5. The oral appliance of claim 4 wherein the lower overmold includes a lower protrusion extending upwardly therefrom that is received in a lower trough defined in the lower base, wherein the lower protrusion comprises a lip and a channel and the lower trough comprises a complementary lip and channel, wherein the lip of the lower protrusion is received in the channel of the lower trough and the lip of the lower trough is received in the channel of the lower protrusion.

6. The oral appliance of claim 1 wherein the right and left hinge members each include at least first, second, third and fourth openings defined therein, wherein the other of the upper member and lower member includes first and second right posts and first and second left posts extending therefrom, wherein the first and second right posts are received in two of the openings in the right hinge member and the first and second left posts are received in two of the openings in the left hinge member, thereby securing the upper member to the lower member, wherein the positioning of the upper member with respect to the lower member can be adjusted by removing the first and second right posts ant: first and second left posts from the respective openings and inserting the first and second right posts and first and second left posts into different openings.

7. The oral appliance of claim 6 wherein the first and second right posts and left first and second left posts each include a flange extending outwardly therefrom.

8. The oral appliance of claim 7 wherein he right and left hinge members extend rearwardly from the upper base and wherein the first and second right posts and the first and second left posts extend upwardly from the lower base.

9. The oral appliance of claim 5 wherein the upper member and the lower member each include a recess defined therein that cooperate to define a central opening that is adapted to receive a portion of a CPAP device.

10. A sleep apnea prevention assembly comprising:
an oral appliance that includes an upper member that includes an upper teeth receiving channel, and a lower member that includes a lower teeth receiving channel, wherein the upper and lower members are hingedly attached to one another such that the upper and lower members are movable between an open position and a closed position, wherein the upper member and the lower member each include a recess defined therein that cooperate to define a central opening when the oral appliance is in the closed position, and a flexible lip seal that extends between the upper member and the lower member, and
a main pipe assembly that comprises a main pipe and a mouth air vent, wherein the main pipe is secured to one of the upper or the lower member and extends through the central opening when the oral appliance is in the closed position, wherein the mouth air vent is positioned proximal of the central opening and has a breathing opening at a proximal end of the main pipe assembly, wherein the main pipe assembly includes an elbow receiver opening at a distal end thereof, and wherein a mouth air path is defined between the breathing opening and the elbow receiver opening.

11. The sleep apnea prevention assembly of claim 10 further comprising a right nose tube assembly having a right nostril opening, and a left nose tube assembly having a left nostril opening, wherein the right and left nose tube assemblies are secured to and in fluid communication with the main pipe assembly, wherein a right nostril air path extends from the right nostril opening through a first aperture in the main pipe and to the elbow receiver opening, and wherein left nostril air path extends from the left nostril opening through a second aperture in the main pipe and to the elbow receiver opening.

12. The sleep apnea prevention assembly of claim 11 wherein the main pipe includes right and left extension members that include right and left circular air openings that are defined by a plurality of extensions that extend between the main pipe and right and left cylindrical receiver members, wherein the right nose tube assembly includes aright lower nose tube that is received on the right extension member and a right upper nose tube in which the right nostril opening is defined extending upwardly from the right lower nose tube, wherein the left nose tube assembly includes a left lower nose tube that is received on the left extension member and a left upper nose tube in which the left nostril opening is defined extending upwardly from the left lower nose tube, wherein the right nostril air path extends from the right nostril opening, through the right upper nose tube, through the right lower nose tube, through the right circular air opening, through the right aperture and to the elbow receiver opening, and wherein the left nostril air path extends from the left nostril opening, through the left upper nose tube, through the left lower nose tube, through the left circular air opening, through the left aperture and to the elbow receiver opening.

13. The sleep apnea prevention assembly of claim 12 wherein the right nose tube assembly includes a right valve that adjusts the flow air between the right nostril opening and the right aperture, and wherein the left nose tube assembly includes a left valve that adjusts the flow air between the left nostril opening and the left aperture.

14. The sleep apnea prevention assembly of claim 13 wherein the right valve comprises a right plug that moves axially within the right circular shaped air opening, and wherein the left valve comprises a left plug that moves axially within the left circular shaped air opening.

15. The sleep apnea prevention assembly of claim 14 wherein the right and left plugs move axially via right and left threaded fasteners, respectively.

16. The sleep apnea prevention assembly of claim 15 further comprising an elbow assembly adapted to be connected to a CRAP machine, wherein the elbow assembly is secured to the distal end of the main pipe assembly,

17. The sleep apnea prevention assembly of claim 10, wherein the flexible lip seal is secured to the upper and lower members by an o-ring that is received in a channel that is defined in the upper member, the lower member and the main pipe.
